(19) United States
(12) Patent Application Publication
(10) Pub. No.: US 2016/0074215 A1
(43) Pub. Date: Mar. 17, 2016

(54) MACULAR SPARING CONTROL METHOD AND CONTROL SYSTEM FOR OPHTHALMIC LASER TREATMENT DEVICE

(71) Applicants: Daoyu ZHOU, Wuhan (CN); Qin DING, Wuhan (CN); Zhongshan CHEN, Wuhan (CN); Yanping SONG, Wuhan (CN); Ming YAN, Wuhan (CN); WUHAN GENERAL HOSPITAL OF GUANGZHOU MILITARY, Wuhan (CN)

(72) Inventors: Yanping SONG, Wuhan (CN); Zhongshan CHEN, Wuhan (CN); Qin DING, Wuhan (CN); Ming YAN, Wuhan (CN); Daoyu ZHOU, Wuhan (CN)

(21) Appl. No.: 14/951,047

(22) Filed: Nov. 24, 2015

Related U.S. Application Data
(63) Continuation of application No. PCT/CN2013/078966, filed on Jul. 8, 2013.

(30) Foreign Application Priority Data
May 25, 2013 (CN) 201310200771.0

(51) Int. Cl. A61F 9/008

(52) U.S. Cl. CPC A61F 9/008; A61F 2009/00863

(57) ABSTRACT

A macular sparing control method and control system for an ophthalmic laser treatment device. The method comprises: during fundus treatment by using a laser treatment device, automatically controlling a switch of the laser treatment device by using a control system; when a laser spot irradiates onto a macular area, stopping excitation of the laser treatment device; and when the laser spot leaves the macular area, starting excitation of the laser treatment device. Damages to macula lutea can be effectively avoided by designing a macular sparing system for laser treatment and another field. During a surgical operation, a surgeon can focus on treatment of an affected part without considering the problem of safety, operation can be performed more efficiently and boldly, and accordingly, the operation efficiency is increased, more comprehensive treatment is realized, and pain of a patient can be relieved to a maximum extent.

### CROSS REFERENCE TO RELATED APPLICATIONS

This application is a bypass Continuation of co-pending Application No. PCT/CN2013/078966, filed on Jul. 8, 2013, for which priority is claimed under 35 U.S.C. §120; and this application claims priority of Application No. 201310200771.0_filed in China on May 25, 2013 under 35 U.S.C. §119, the entire contents of all of which are hereby incorporated by reference.

### FIELD OF THE INVENTION

The present invention relates to the field of ophthalmic laser therapy equipment, which more specifically is a macular sparing control method and control system for ophthalmic laser treatment device with high safety.

### DESCRIPTION OF RELATED ART

As shown in FIG. 2, an anatomy of normal fundus mainly contains optic papilla, macula lutea, optic disc, retinal blood vessels system, etc. Optic papilla is made up of optic nerve fibers which is the part of the optic nerve goes through the wall of eyeball. Optic papilla generally appears orange. A sunken part where optic nerve fibers converge in central optic papilla is called optic disc. Optic papilla is the most significant symbol for observing fundus image because it is easily reflective. As the most sensitive part of vision, macula lutea is rich in pigment epithelium where locates in the central posterior pole of eyeball. Macula lutea is the darkest part of eyeball's posterior pole which appears red or rufous, it is nearly circular. There is also a small sunken part in central macula lutea which is called fovea. The fovea is the most sensitive part of vision, it can reflect light to form a very small light point during fundus examination. Retinal blood vessels system contains central retinal artery, central retinal vein, cillio-retinal artery and cillio-retina vein, each of them has multi branch to supply nutrition for retina. Retinal blood vessels system is one of the most important indexes for evaluating the fundus image processing result. Within the region of upper hemal arch and lower hemal arch is a broadly defined macular area. The macular area is the most sensitive part of vision.

The said macula lutea locates in the central posterior pole of eye. As the most sensitive part of vision, damage to macula lutea will cause irreversible functional injury. Laser is a highly effect treatment for fundus diseases, but the macular area is a restricted zone of laser treatment because it will cause serious damage to visual function if the macular area is accidentally damaged by laser. Therefore, surgeon must operate a laser treatment device very cautiously during ophthalmic laser operation, in order to avoid damaging the macular area. But the difficulty of operation will be quite high when some of the affected parts are very near to the macular area. If a surgeon operates conservatively, he may leave affected parts uncured, if a surgeon operates boldly, he may accidentally damage the macular area. Consequently, surgeon's experience is tested to the utmost during such operation. Different therapeutic effects from different surgeons are prejudicial to objective risk evaluation of surgical equipment.

### SUMMARY OF THE INVENTION

The present invention has an object to overcome the above problems and to provide a macular sparing control method and control system for ophthalmic laser treatment device.

The aforementioned object is achieved by the following technical solutions.

The present invention firstly discloses a macular sparing control method for ophthalmic laser treatment device. During a treatment process of fundus by a laser treatment device, a control system automatically controls the switch of the said laser treatment device. It stops excitation of the laser treatment device when the laser spot irradiates onto the macular area, and starts excitation of the laser treatment device when the laser spot leaves the macular area. In laser treatment and other fields, the design of macular sparing system can avoid macula lutea damage effectively. Then, the recognition can be understood from the aforementioned anatomy that the broadly defined macular area is formed by a region within upper hemal arch and lower hemal arch, the said region is the most sensitive part of vision, which is also easily being accidentally damaged during operation. Therefore, to secure the safety of the said region, we just need to stop excitation of the laser treatment device when the laser spot irradiates onto it. Furthermore, the stop of excitation of the laser treatment device is automatically controlled by a control system which doesn't need manual operation. In consequence, surgeon can concentrate on the treatment of affected parts without considering safety problems, operates more efficiently and boldly; the operation efficiency is increased, more comprehensive treatment is realized, and pain of a patient can be relieved to a maximum extent.

The aforementioned method comprises steps of:

S1 Collect a fundus image. The fundus image can be collected by existing fundus photographic camera or video camera, which is the regular technical means in current ophthalmology.

S2 Recognize a macular area on the fundus image. According to ophthalmology-anatomy, macula lutea is the darkest part of posterior pole which appears red or rufous, it has a nearly circular shape, the broadly defined macular area is formed by a region within upper hemal arch and lower hemal arch, the said region is the most sensitive part of vision. To secure the safety of operation, the broadly defined macular area should be defined as macular area. Therefore, the region within upper hemal arch and lower hemal arch is the macular area in present invention.

S3 A control system monitors the movement of the laser spot on the fundus image. The principle of present invention is to avoid the laser spot irradiates onto the said macular area, so that the damage to macular area during operation can be avoided. Therefore, the position of the laser spot light on the fundus is crucial, it needs to be monitored in real time. For this reason, the present invention implements real time monitoring through a high frequency capture of the laser spot's position on fundus image by a control system

S4 Stop excitation of the laser treatment device when the laser spot irradiates onto the macular area, and start excitation of the laser treatment device when the laser spot leaves the macular area. In present invention, a camera collects a fundus image, recognize a macular area after analytic processing the fundus image. When the laser spot irradiates onto the said area, the laser treatment device is triggered to enter a standby status automatically, so that the laser can't be excitated, the damage to macula lutea is prevented at the same time.

The principle of the said method shows that the switch of the laser treatment device is automatically controlled by monitoring the movement of the laser spot through a control system. Surgeon can concentrate on treatment without considering the control of the laser spot's movement during operation, at the same time, with an automatic protection from the system, the macular area will be assured that it won't be irradiated by the laser spot during operation. Then surgeon can operate more boldly, and deliver a secure operation onto the region which is extremely near to the macular area without having worries about any accidentally damage to the macular area by misoperation. Thereout, a thorough treatment is realized, the operation efficiency is increased significantly, and pain of a patient will be relieved to a maximum extent. On the other hand, the said method avoids the operation risks caused by human error, makes the risk of operation as ophthalmic laser treatment be evaluated objectively, promotes the application and popularization of the said operation method.

During the auto-reorganization of the fundus image, a certain differences of collected images will be caused by the brightness of light, the length of distance, angle and other possible reasons. To further improve the safety of operation, the said fundus image in the said step S2 collected during the recognition of a macular area on the fundus image is converted to a binary image. The aforementioned pre-processing aims at eliminating the noise of the image, converting the image to a clear two points line graph with one pixel, so that the characters of the fundus image are accurately extracted

The said step S2 comprises steps of:

S21 Enhance image contrast

S22 Perform a further filtering process on the image which contrast has been regulated;

S23 Perform a hole filling process on the filtered image;

S24 Perform a thinning process on the hole filled image.

The said step S3 of present invention has many applicable embodiments, which preferred comprises steps of:

S31 Collect an image which contains the position of the laser spot on the fundus

S32 Recognize the position of the laser spot

S33 Perform a superimposed-contrast between the image which the laser spot position has been recognized and the image which the macular area has been recognized in step S2, and judge whether there is a superimposition of the spot and the macular area. If there is a superimposition, it will judge that the spot irradiates onto the macular area, otherwise, it will judge that the spot doesn't irradiate onto the macular area.

Repeat steps from S31 to S33 in high frequency.

In order to further secure the safety of operation, the sampling time interval can be controlled at millisecond level through collecting images in high frequency.

The said step S3 can also comprises preferred steps of:

S310 Continuously collect images which contain the position of the laser spot on the fundus

S320 Recognize positions of the laser spot on continuous images, to form a trajectory of the laser spot;

S330 Predict the position of the laser spot on the next fundus image based on the said trajectory;

S340 Judge whether the said position of the laser spot enters the recognized macular area. If it enters, it will judge that the spot irradiates onto the macular area, otherwise, it will judge that the spot doesn't irradiate onto the macular area.

Repeat steps from S310 to S340 in high frequency.

To further improve the safety of operation, the present invention discloses a circular pre-warning area set outside the said macular area, the control system sounds the alarm when the laser spot irradiates onto the pre-warning area. Therefore, surgeon can be more carefully when operates nears the macular area with the auto-notification of the system, possible risks brought by the misoperation of automatic control of device are avoided.

The way of forming the said pre-warning area is:

S5. Isometrically extend the macular area outward 1˜2 mm to form a pre-warning area

During the operation, a pre-warning area which has a size of 1˜2 mm is sufficient to arouse a surgeon's awareness. In addition, the size of the said pre-warning area can't be too large because the size of fundus is not big, at the same time, a pre-warning area which size is too big will seriously affect the operation of surgery. Through experiments, the best range of the pre-warning area's size is controlled at lower than 1 mm.

The present invention further discloses a macular sparing control system for ophthalmic laser treatment device, which mainly comprises of:

A laser treatment device;

A fundus image acquisition device;

A control system used for processing images, monitoring the movement of the laser spot and automatically controlling the switch of the laser treatment device;

The said laser treatment device and the said fundus image acquisition device are selected from the art. The improvement of the present invention is that the said control system is added with the said control method which is used for processing images, monitoring the movement of the laser spot and automatically controlling the switch of the laser treatment device

The present invention further discloses an improvement of the laser treatment device in the art, which is that employing 810 nm semiconductor laser for micropulse threshold photo-coagulation, the said 810 nm semiconductor laser has:

Laser pulse width: 0.15˜0.25 ms;

Laser slot time: 1.6˜2.2 ms;

Load factor: 8%˜12%

810 nm semiconductor laser belongs to near infrared, which can penetrates tissues more deeply to give a better laser treatment effect. Moreover the main pigment of macula lutea is lutein, which doesn't absorb 810 nm semiconductor laser, therefore, the said laser can easily penetrates retina in the macular area, mainly works at purpurogenous membrane, leaves minor losses of optic cells, avoids damage to the macular area in a certain extent.

The classic laser treatment modes include a long pulse mode (pulse width is 30 min) and continuous wave mode (pulse width is 200 ms). The said long pulse mode is a long time photothermal effect on retina, which causes a huge damage to macular tissues. Therefore, the present invention selects micropulse, which is that the laser's pulse width (time of pulse) reaches to millisecond level. The present invention has preferred parameters as follows: laser pulse width: 0.15˜0.25 ms; laser slot time: 1.6˜2.2 ms; load factor: 8%˜12%. In this way, the sum of each treatment cycle's exposure time is at some percentage of a long pulse mode's time. For the laser of each pulse works in a very short time, its absolute exposure dose is smaller than the damage threshold, the photocoagulation temperature of subliminal value is smaller than 20° C. Because each micropulse has a longer slot time, retina has a sufficient time to diffuse and reduce heat inside with a smaller heat accumulation, the heat damage is reduced at mean time, only potential damage (sub-inactivation) to fundus occurs.

In conclusion, the present invention provides a macular sparing control method through improving the control method of ophthalmic laser treatment device. The macula lutea is protected by means of setting the macular area as laser restricted zone, and controlling the laser spot stops irradiation when it moves to the said zone. The said recognition of whether the laser spot irradiates onto the macular area and the control of whether the laser treatment device is activated are all controlled by the control system automatically. Surgeon can concentrate on treatment without considering the said recognition and the said control during operation, the operation efficiency is highly increased, and pain of a patient can be relieved to a maximum extent. Based on the aforementioned method, the present invention also gives improvements of recognition rate, sampling matching algorithms, pre-warning area, etc., which further improve reliability of the system. Among the laser treatment devices which employ the method disclosed by the present invention, the present invention also gives further improvements of laser's type, parameterized treatment mode, etc., which can all improve the safety of operation.

### BRIEF DESCRIPTION OF THE DRAWINGS

FIG. 1 is a flow chart of the first embodiment of the present invention

FIG. 2 is an original fundus image collected by laser treatment device

FIG. 3 is a flow chart of pre-processing of the image shown in FIG. 1 by the first embodiment of the present invention

FIG. 4 is the fundus image pre-processed by the first embodiment of the present invention

FIG. 5 is a fundus image collected when the laser spot irradiates on macular area

FIG. 6 is a fundus image captured when the laser spot leaves the macular area

FIG. 7 is a flow chart of monitoring the movement of the laser spot on the fundus by a control system in the second embodiment of the present invention

FIG. 8 is a flow chart of monitoring the movement of the laser spot on the fundus by a control system in the third embodiment of the present invention

FIG. 9 is the fundus image pre-processed by the fourth embodiment of the present invention

### DETAILED DESCRIPTION OF THE PREFERRED EMBODIMENTS

A further description of the present invention will now be given referring to the accompanying drawings. The said drawings only give exemplary description, which couldn't be understood as restriction for the present invention. Some of the steps in the said drawings will be omitted or be replaced by simple words for better illustrating the embodiments of the present invention, which doesn't means that the control method could be so rough. A person skilled in the art would understand that some public known steps in the said drawings are omitted.

### Embodiment 1

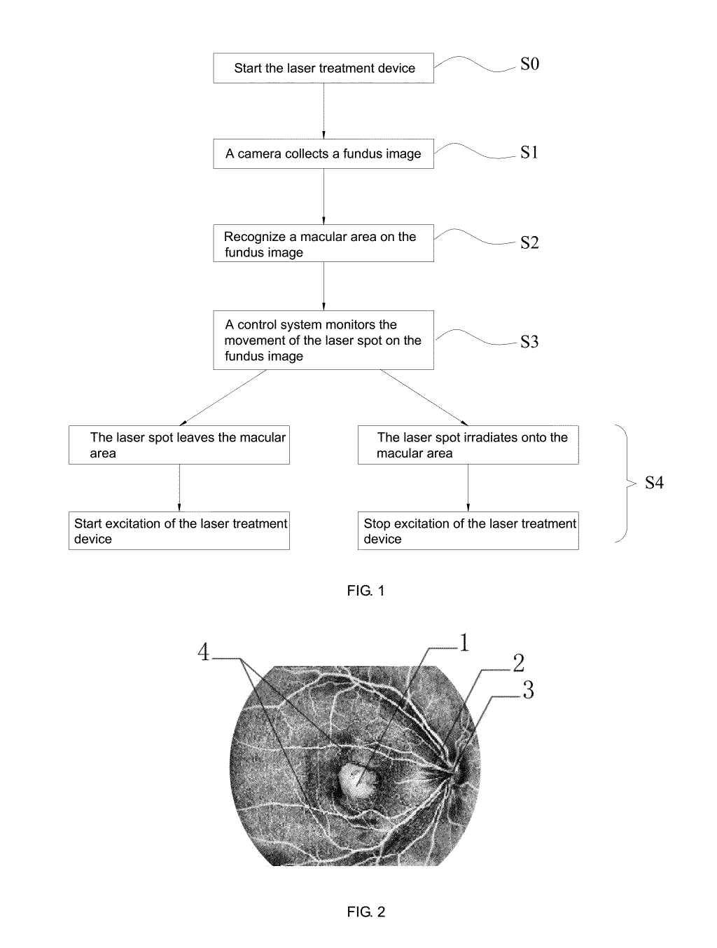

A camera collects a fundus image, recognize a macular area after analytic processing. When the laser spot irradiates onto the said area during operation, the laser treatment device will be triggered to enter a standby status automatically, so that the laser can't be excitated, damage to macula lutea is prevented at the same time. The said control method's flow chart is shown in FIG. 1, which comprises steps of:

S0. Start the laser treatment device;

S1. A camera collects a fundus image;

S2 Recognize a macular area on the fundus image

S3. A control system monitors the movement of the laser spot on the fundus image

S4. Stop excitation of the laser treatment device when the laser spot irradiates onto the macular area, start excitation of the laser treatment device when the laser spot leaves the macular area.

During the auto-recognition process of the fundus image, the image of retina read by a camera or a photographic camera of the laser treatment device in step 2 is affected by the brightness of light, the length of distance, angle and other possible reasons. From the reversed gray level image of retina shown in FIG. 2, we can clearly pick out macula lutea 1, optic papilla 2, optic disc 3, retinal blood vessels system 4, etc. The said collected original image needs pre-processing because it is not a language that a computer can recognized.

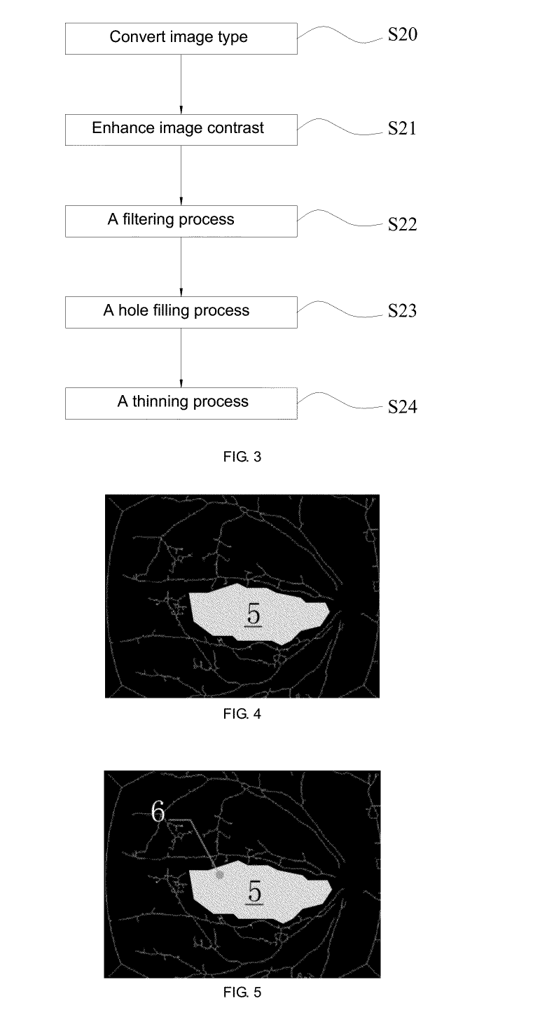

The pre-processing shown in FIG. 3 comprises the following steps of:

S20 Convert image type

S21 Enhance image contrast

S22 Perform a further filtering process on the image which contrast has been regulated;

S23 Perform a hole filling process on the filtered image;

S24 Perform a thinning process on the hole filled image.

The CPU of the laser treatment device has limited computing power because it is mostly SCM. A violent transformation of the image is initiated by compressing the image and eliminating useless information for improving computer efficiency. Next, the characters of retina which contain macula lutea 1, optic papilla 2, optic disc 3, retinal blood vessels system 4, etc, are highlighted through enhancing the retina image contrast. After enhancing the retina characters, a filtering process can start which aims at eliminating the noise of the image through converting the image to a clear two points line graph with one pixel, the characters of fundus image are accurately extracted. The region within the upper hemal arch and lower hemal arch recognized by computer is the broadly defined macular area which is the most sensitive part of vision. Then perform a hole filling process on inside and outside of the said macular area: inside of the said macular area is mainly filled with black (keep information of retinal blood vessels system in order to facilitate surgeon's operation). At last, perform a thinning process on the image for enhancing the macular area, and set up the monitoring area. The final processed image is shown in FIG. 4: a computer can fast calculate and recognize the monitoring area 5 which is the said macular area through the clear two points line graph with one pixel. The said pre-processing is the first step that the retina image recognition system runs, which is also the foundation of the retina recognition, it directly affects the result of retina recognition.

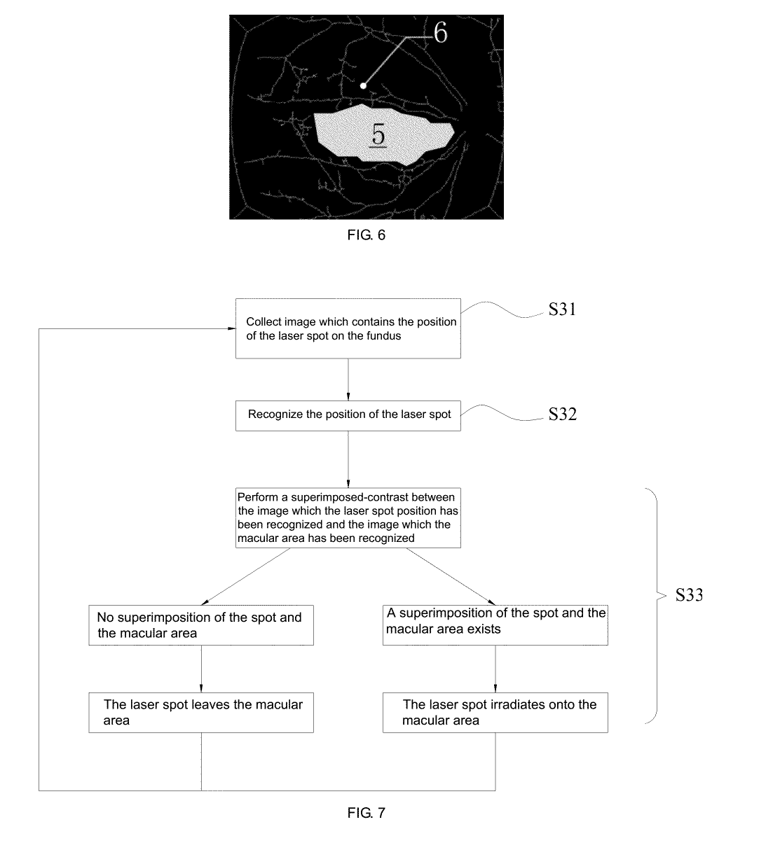

The technique for monitoring the movement of the laser spot on the fundus by a control system in the said step S3 is the image surveillance technique known in the art. As shown in FIG. 5, a control system stops excitation of the laser treatment device when the laser spot 6 irradiates onto the macular area 5. As shown in FIG. 6, the said control system starts excitation of the laser treatment device when the laser spot 6 leaves the macular area 5.

### Embodiment 2

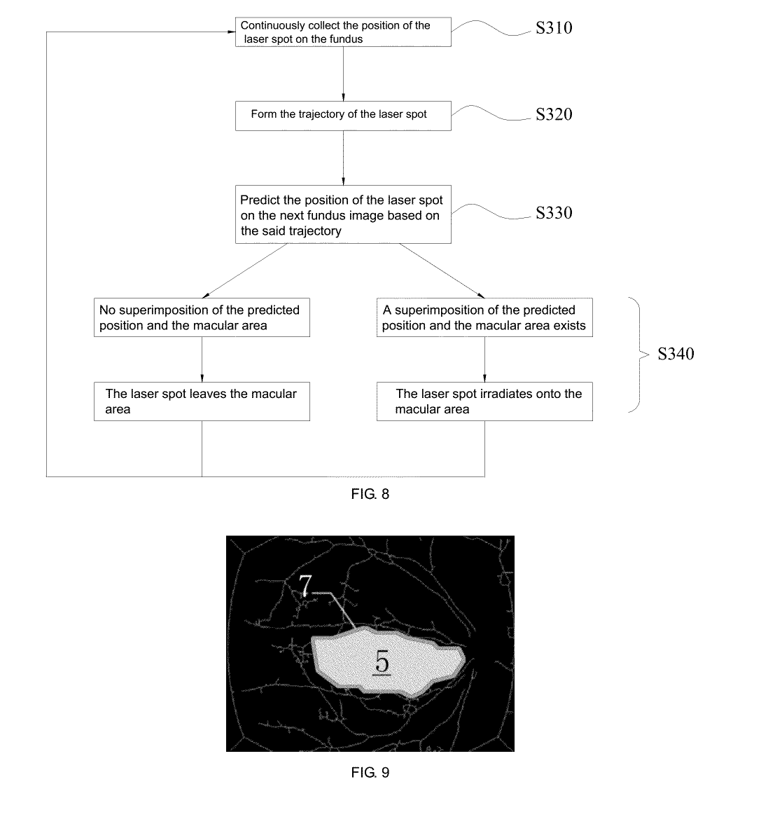

The second embodiment has a same basic control procedure as the first embodiment, with a main difference at step S3. The step S3 of the said second embodiment discloses an improvement of the image surveillance technique known in the art. The second embodiment shown in FIG. 7 comprises steps of:

S31 Collect image which contains the position of the laser spot on the fundus;

S32 Recognize the position of the laser spot

S33 Perform a superimposed-contrast between the image which the laser spot position has been recognized and the image which the macular area has been recognized in step S2, and judge whether there is a superimposition of the spot and the macular area. If there is a superimposition, it will judge that the spot irradiates onto the macular area, otherwise, it will judge that the spot doesn't irradiate onto the macular area.

Repeat steps from S31 to S33 in high frequency.

In order to further secure the safety of operation, the sampling time interval can be controlled at millisecond level through collecting images in high frequency.

### Embodiment 3

The third embodiment has a same basic control procedure as the first embodiment, with a main difference at step S3. The step S3 of the said third embodiment discloses an improvement of the image surveillance technique known in the art. The third embodiment shown in FIG. 8 comprises steps of:

S310 Continuously collect images which contain the position of the laser spot on the fundus;

S320 Recognize positions of the laser spot on continuous images, to form a trajectory of the laser spot;

S330 Predict the position of the laser spot on the next fundus image based on the said trajectory;

S340 Judge whether the said position of the laser spot enters the recognized macular area. If it enters the recognized macular area, it will judge that the spot irradiates onto the macular area, otherwise, it will judge that the spot doesn't irradiate onto the macular area.

Repeat steps from S310 to S340 in high frequency.

### Embodiment 4

The fourth embodiment has a same basic control procedure as the first embodiment. The present invention discloses a circular pre-warning area 7 set outside the said macular area 7. As shown in FIG. 9, the control system sounds the alarm when the laser spot irradiates onto the pre-warning area 7. The said pre-warning area 7 is formed by extending the macular area outward 1 mm.

The aforementioned relationship between position and steps described in the said drawings are exemplary description, which can't be the limitation of the present invention. Apparently, the said embodiments are only designed as examples for disclosing a clear description for the present invention, which can't limit any embodiments of the present invention. To the person with ordinary skill in the art, other modifications and variations are possible in light of the said description. Therefore, the said embodiments are not exhaustive for the present invention. Any variations, modifications or improvements can be made within the spirit and principles of the present invention and shall be within the protection area of the present invention.

1. A macular sparing control method for ophthalmic laser treatment device, wherein that a control system automatically controls the switch of the said laser treatment device during the treatment process of fundus by the said laser treatment device: stops excitation of the laser treatment device when the laser spot irradiates onto the macular area, and starts excitation of the laser treatment device when the laser spot leaves the macular area.

2. The macular sparing control method for ophthalmic laser treatment device according to claim 1, wherein the said method comprises steps of:
S1 collect a fundus image;
S2 recognize a macular area on the fundus image;
S3 a control system monitors the movement of the laser spot on the fundus image; and
S4 stop excitation of the laser treatment device when the laser spot irradiates onto the macular area, start excitation of the laser treatment device when the laser spot leaves the macular area.

3. The macular sparing control method for ophthalmic laser treatment device according to claim 2, wherein the said fundus image in the said step S2 collected during the recognition of the macular area on the fundus image will be converted to a binary image.

4. The macular sparing control method for ophthalmic laser treatment device according to claim 3, wherein the said step S2 comprises steps of:
S21 enhance image contrast;
S22 perform a further filtering process on the image which contrast has been regulated;
S23 perform a hole filling process on the filtered image; and
S24 perform a thinning process on the hole filled image.

5. The macular sparing control method for ophthalmic laser treatment device according to claim 2, wherein the said step S3 comprises steps of:
S31 collect image which contains the position of the laser spot on the fundus;
S32 recognize the position of the laser spot;
S33 perform a superimposed-contrast between the image which the laser spot position has been recognized and the image which the macular area has been recognized in step S2, and judge whether there is a superimposition of the spot and the macular area. If there is a superimposition, it will judge that the spot irradiates onto the macular area, otherwise, it will judge that the spot doesn't irradiate onto the macular area; and
repeat steps from S31 to S33 in high frequency.

6. The macular sparing control method for ophthalmic laser treatment device according to claim 2, wherein the said step S3 comprises steps of:
S310 continuously collect images which contain the position of the laser spot on the fundus;
S320 recognize positions of the laser spot on continuous images, to form a trajectory of the laser spot;
S330 predict the position of the laser spot on the next fundus image based on the said trajectory;
S340 judge whether the said position of the laser spot enters the recognized macular area. If it enters, it will judge that the spot irradiates onto the macular area, otherwise, it will judge that the spot doesn't irradiate onto the macular area; and
repeat steps from S310 to S340 in high frequency.

7. The macular sparing control method for ophthalmic laser treatment device according to claim 1, wherein a circular pre-warning area is set outside the said macular area, the said control system sounds the alarm when the laser spot irradiates onto the pre-warning area.

8. The macular sparing control method for ophthalmic laser treatment device according to claim 7, wherein the way of forming the said pre-warning area is:
S5 isometrically extend the macular area outward 1˜2 mm to form a pre-warning area.

9. A macular sparing control system for ophthalmic laser treatment device which employs the control method of claim 1, comprising:
a laser treatment device;
a fundus image acquisition device; and
a control system for processing images, monitoring the movement of the laser spot and automatically controlling the switch of the laser treatment device.

10. The said control system according to claim 9, wherein the said laser treatment device employs 810 nm semiconductor laser for micropulse threshold photo-coagulation, the said 810 nm semiconductor laser has:
laser pulse width: 0.15˜0.25 ms;
laser slot time: 1.6˜2.2 ms;
load factor: 8%˜12%.
